(19) United States
(12) Patent Application Publication
(10) Pub. No.: US 2012/0282011 A1
(43) Pub. Date: Nov. 8, 2012

(54) WEARABLE HAND SANITIZER DISPENSER

(76) Inventor: Gregory Francois, Cambria Heights, NY (US)

(21) Appl. No.: 13/102,037

(22) Filed: May 5, 2011

(51) Int. Cl. B43K 8/00; B67D 7/84

(52) U.S. Cl. 401/196; 222/175

(57) ABSTRACT

A wearable hand sanitizer dispenser that is user friendly, leak-proof, and cost-effective is disclosed. The wearable hand sanitizer dispenser comprises a sponge enclosed in a silicone case which then is placed in a pocket located on a wristband. The silicone case has a plurality of self-sealing slits to control release of sanitizer and a plurality of refill apertures for allowing the user to fill it up with a large quantity of sanitizer or the like. The pocket also contains a plurality of openings to control release of sanitizer. When the pocket on the wristband is not compressed, the sponge inside the silicone case absorbs sanitizer so that inadvertent fluid leakage will not occur.

### REFERENCE TO RELATED APPLICATIONS

This patent application claims the benefit of U.S. Provisional Application No. 61/331,381 filed on May 5, 2010, the disclosure of which is incorporated herein by reference in its entirety.

### BACKGROUND OF THE INVENTION

1. Field of the Invention

The present invention relates to a sanitizer dispenser, and more particularly to a wearable hand sanitizer dispenser that is easy to use, leak-proof, refillable, and cost-effective.

2. Description of Related Art

A hand sanitizer or hand antiseptic is a supplement or alternative to hand washing with soap and water. Hand sanitizers present a way to reduce the spread of infection. When hands are not visibly dirty, the United States Centers for Disease Control and Prevention and many other world public health authorities recommend alcohol based (such as ethanol, isopropanol, n-propanol at no less than 70 percent) hand sanitizers for the most effective germicidal efficacy as an acceptable alternative to soap and water for hand hygiene.

Although there are a lot of hand sanitizers that are packaged in sanitization stations or in large containers they are not portable and handy to be used outdoors, especially in social occasions, playgrounds or in the markets. Even when a bottle of hand sanitizer is brought in a purse or a bag, if there are any suspicion that the hand may have been in contact with germs, it is unwise to open a bag or purse to take out the bottled sanitizer because the contaminated hand would spread germs to more surfaces.

Moreover, a bottle of sanitizer may spill when a user attempts to open it. Therefore, it is obvious that there is a need for a device that allows a person to have convenient, easy direct access to a hand sanitizer that can be worn on the body, specifically the wrist.

Various methods of wearable hand sanitizers have been disclosed in the art. More specifically, by way of example, U.S. Pat. No. 7,285,114 to Harper (2007) discloses a hand sterilizing apparatus and method promoting the convenient use of hand sterilizing fluids and gels applied to reduce the hand-borne transmission of pathogens. The invention comprises at least two flexible blister wells, each said blister well adapted to retain and preserve a single application amount of hand sanitizing fluid. The blister surface can be simply weakened by thinning a designated area of the blister wall during formation of the well. Another form of release is a group of release points characterized by a self-closing slit or puncture opening. The invention does not have apertures allowing for refill of the hand sanitizer. The invention does not provide mechanism in addition to self-closing slit such as a sponge type of absorbent that can absorb the hand sanitizer to control the quantity to release or prevent sanitizer from dripping if compressed inadvertently.

U.S. PreGrant Pub. Nos. 2004/0111071; 2004/0162534; 2005/0124945; 2006/0091156; 2007/0088298; 2008/0067193; 2008/0230560; 2010/0314414; U.S. Pat. Nos. 7,135,011; 7,316,332; and PCT Pub. No. WO 2004/052425 to Powers et al. discloses wearable skin treatment devices including disinfectant, sunscreens, medications, and moisturizing lotions. The invention comprises dispersions in the form of neck-worn and disposable wrist-worn devices. The invention utilizes complex releasing mechanisms to control release and to prevent the sanitizer from dripping. For the simplest model, the invention involves a check valve to prevent the sanitizer from dripping. The invention does not have a sponge type absorbent that can absorb the hand sanitizer to prevent the sanitizer from dripping or to control the quantity of release. Check valve or diaphragm valve are still more complex than self-sealing slit, and thus contributing additional cost to manufacture.

U.S. PreGrant Pub. No. 2006/0078484 to Greep (2006) discloses a hand sanitizing device that may be configured in the form of any suitable shape of absorbent material that is saturated or impregnated with a hand sanitizing liquid material and retained within a housing of the device such that when the saturated material is released or exposed from the housing, the saturated material can be contacted with the surface to be cleaned and then retracted into or covered by the housing. The invention does not provide means that allows for refill of the hand sanitizer.

Overall, there still exists a need to have a wearable hand sanitizer device that is leak proof, refillable, easy to use, and simply configured, can hold large quantity of hand sanitizer, can be constructed and manufactured at low cost.

### SUMMARY OF THE INVENTION

The objective is to provide a hand sanitizer device that is portable and can be easily accessed without contaminating other surfaces.

The objective is to provide a hand sanitizer device that has a simple configuration, to achieve simplicity of construction, and is easy to use.

The objective is to provide a hand sanitizer device that is leak proof without complex mechanical construction.

The objective is to provide a hand sanitizer device that is refillable.

The objective is to provide a hand sanitizer device that is cost-effective to manufacture.

The objective is to provide a hand sanitizer device that can hold a large quantity.

The device of the present invention is a sanitizer dispenser. It works by allowing a person to fill it up with a large quantity of hand sanitizer or the like. It primarily serves as a means for a person to clean their hands with use of hand sanitizer liquids after being in contact with suspicious source of germs. It may be in a form of a wristband and/or gloves. A preferred embodiment of the present invention is a wristband, which comprises an elongated body member with holes that allow for ventilation; a pocket for holding hand sanitizer reservoir is located on the elongated body, either removably attached to the elongated body or being an integral part of the elongated body, having a plurality of openings for releasing sanitizer onto hands; a reservoir, wherein a sponge is retained, having a plurality of self-sealing slits for controlling release of sanitizer and a plurality of refill apertures for refilling of sanitizer; and an interjecting means for interjecting sanitizer into the reservoir. The reservoir is preferably made of silicone polymer; other elastomers suitable for the intended purpose known in the art may be used.

In one embodiment, the locking and releasing mechanism to control release of the sanitizer is a simple self-sealing slit on the silicone case. The silicone case has a plurality of self-sealing slits. The sponge is saturated in hand sanitizer and may be re-saturated after depletion by interjecting hand sanitizer through apertures up to 150 mL. The pocket has a plurality of openings where the hand sanitizer can be released onto a person's hand. The silicone polymer has a self-sealing physical property. The advantage of a self-sealing slit is that it is simple and inexpensive to manufacture. However, if the release of the hand sanitizer is only controlled by self-sealing slits, then when the pressure in the reservoir is high, there is a possibility of a leak. Due to the nature that the sponge can absorb the sanitizer, it can serve as release control and can prevent hand sanitizer from dripping due to gravity. The sponge impregnated with hand sanitizer gradually releases the hand sanitizer when it is squeezed, which then passes through the slit on the silicone coating and through the openings on the pocket where the hand sanitizer can be released, and onto a person's hand.

Sponges are commonly made from cellulose plant fibers, or foamed plastic polymers or natural sponges. Among available synthetic sponges, PVA (Polyvinyl Alcohol) is the most dense, highly absorbent material. PVA sponge can absorb up to 12 times its dry weight in water. When saturated with water, the wet volume is about 20% greater than the dry volume. Other sponge and other absorbent materials may also be used for the same purpose to absorb and retain hand sanitizer inside the silicone case to control release and to prevent the sanitizer from leaking.

The reservoir, which encases the sanitizer saturated sponge, may be made of silicone rubber or other material known in the art that are suitable for the intended purpose. Silicone rubber, a mixture of silicon together with hydrogen, oxygen, is elastomeric and self-sealing so that the slit can be self-sealed to prevent sanitizer from leaking.

The elongated body which is the major part that is worn on the body may be made of plastic, neoprene, silicone polymer, and/or other materials suitable for the intended purpose known in the art. The pocket that may be attached to the elongated body of the wristband or may be an integral part of the elongated body can be made of the same material as the elongated body. The pocket has a plurality of openings for releasing the sanitizer. In one embodiment, the openings on the pocket have no cap or seal so once the sanitizer passes through the slit on the silicone case, it may come out of the pocket without any control. In another embodiment, the openings are in fact self-seal pre-slit like the ones on silicone case. This mechanism provides additional leak proof.

In use, the user uses the hand that is free of the wristband to compress the pocket on the wristband to release the hand sanitizer from the reservoir. Or the user can rub the pocket of the wristband on the palm or the back of the hand free of wristband to release hand sanitizer from the reservoir.

In another embodiment, there may be more complex locking and releasing mechanism that helps to control release of sanitizer and to prevent the sanitizer from leakage. These complex locking and releasing mechanism may include but is not limited to diaphragm valves or check valves which are controlled by various types of actuators. In another embodiment, the refill apertures may include other closing mechanisms such as a cap. In another embodiment, the pocket may be a pouch like receptacle with an opening to insert and take out the silicon encased sponge. In another embodiment, the pocket may also have a plurality of apertures for interjecting sanitizers and caps for leak proof.

In another embodiment that takes the form of a glove. A pocket may be located on the top of the glove. The sponge saturated with sanitizer is located within the silicone rubber which is then located within the pocket. The pocket may be made of the same material that is used in the wristband. The glove is made of the typical materials used for gloves and may or may not have ventilation holes.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a front perspective view of a wristband embodiment of the present invention;

FIG. 2 is a top view of an embodiment of the present invention;

FIG. 3 is a side view, where a pocket is shown, of an embodiment of the present invention; and

FIG. 4 is a front perspective view of a glove embodiment of the present invention

### DESCRIPTION OF THE PREFERRED EMBODIMENT

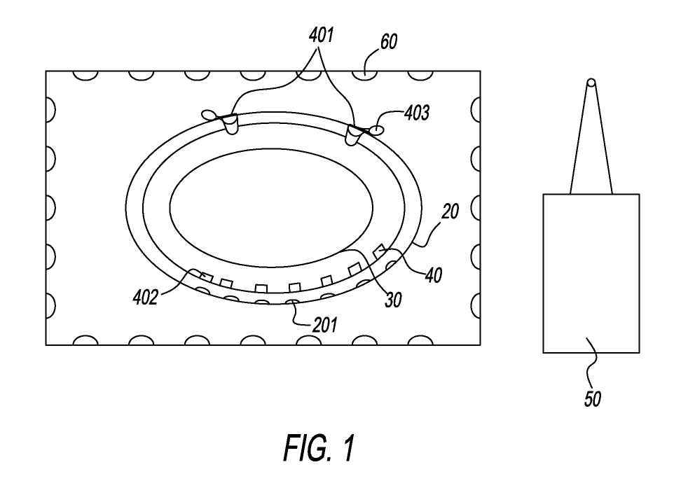

Referring to FIG. 1, there is disclosed a front view of a wristband embodiment according to the present invention. The embodiment 1 comprises an elongated body member 10 with holes 60 that allow for ventilation; a pocket 20 contained on the elongated body 10, either is removably attached to the elongated body 10 or is an integral part of the elongated body 10; a sponge 30 encased in silicone casing 40 with two apertures 401 on the top. The sponge 30 is saturated in hand sanitizer and may be re-saturated after depletion by interjecting hand sanitizer through apertures 401 on the silicone case 40 and/or pocket 20 using interjecting device 50. The refill apertures 401 may have caps 403 to prevent leakage. The silicone case 40 also has a plurality of openings with self-sealing slits 402 to control release and leakage of sanitizer. The pocket 20 has a plurality of openings 201 where the hand sanitizer can be released onto a person's hand. The openings 201 on the pocket may also be self-sealing pre-slits thus providing further leakage proof mechanism. The silicone polymer has a self-sealing physical property. The advantage of a self-sealing slit is that it is simple and cost-effective to manufacture. However, if the release of hand sanitizer is only controlled by self-sealing slits 402 and/or 201 when the pressure inside the reservoir (silicone case) is high, it may possibly leak.

The function of sponge 30 can absorb liquid sanitizer and serve as release control and can prevent hand sanitizer from dripping/leakage due to gravity. The sponge 30 impregnated with hand sanitizer when squeezed releases the sanitizer through the slits 402 on the silicone case 40 and the openings 201 on the pocket 20 onto the user's hand. While it is not squeezed, the sanitizer is absorbed by the sponge 30 so that inadvertent fluid leakage will not occur.

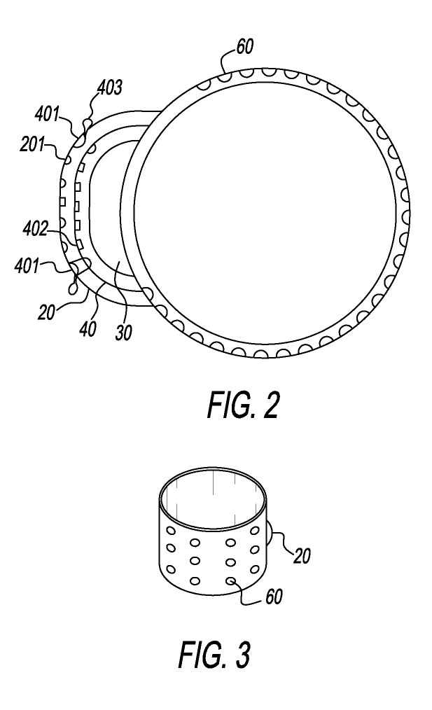

FIG. 2 is a top view of a wristband embodiment of the present invention. On the left hand side of the top view of the wristband 1 there is a pocket 20 having a plurality of openings 201 for releasing the hand sanitizer. The diameter of the wristband 1 plus the height of the pocket 20 is about 2.23 inches. The height of the pocket 20 is about 0.27 inch. A sponge 30 saturated with sanitizer is located with a silicone case 40 which is inside the pocket 20. There are also a plurality of refill apertures 401 on the silicone case 40 and/or pocket 20 and a plurality of release openings 402 and 201 on the silicone case and pocket 20.

FIG. 3 is a side view of a wristband 1 where a pocket 20 is shown with two dimensions. The width of the elongated body 10 is about 1.13 inches. The pocket 20 is in an ellipse shape with about 1.10 inches in length and about 0.71 inch in width. The height of pocket 20 is about 0.27 inch (from FIG. 2).

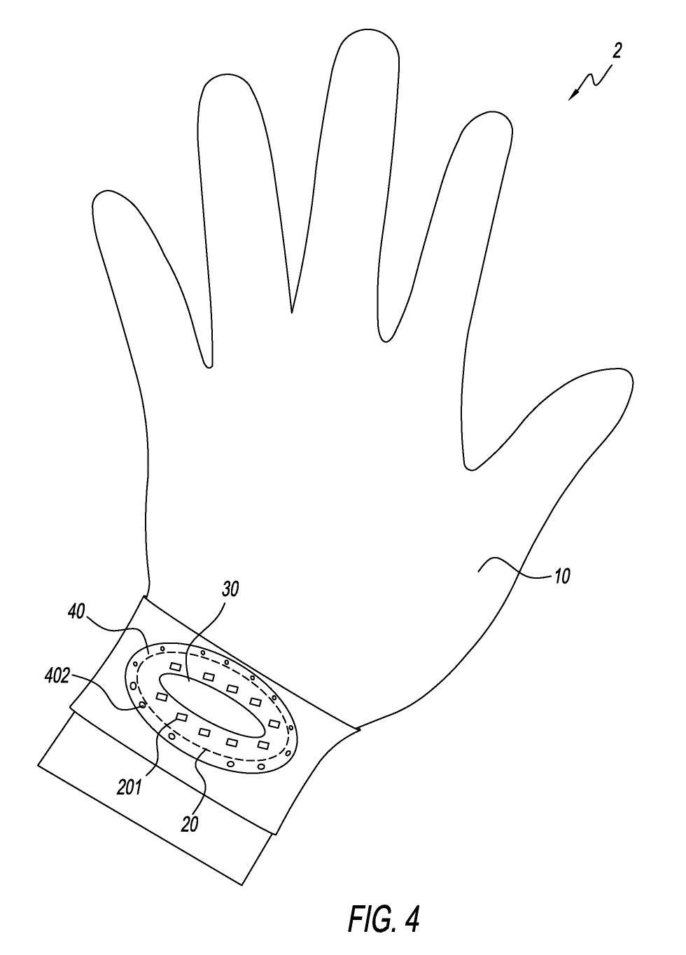

Referring to FIG. 4 for another embodiment of sanitizer device that takes the form of a glove 2, a sanitizer saturated sponge 30 is enclosed within a silicone case 40, which is then located inside a pocket 20 on the glove 2. The pocket 20 may be made of the same material that is used in the wristband 1. The glove 2 is made of the typical materials used for the gloves.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A wearable hand sanitizer dispenser comprising:
a. a body member with holes;
b. a pocket located on the body member, the pocket having a plurality of openings for controlling release of sanitizer;
c. a case located within the pocket, the case having a plurality of openings for controlling release of sanitizer and a plurality of refill apertures for refilling of sanitizer;
d. an absorbent material enclosed in the case for absorbing sanitizer; and
e. an interjecting means for interjecting sanitizer into the case.

2. The wearable hand sanitizer dispenser of claim 1, wherein the absorbent material is a sponge.

3. The wearable hand sanitizer dispenser of claim 1, wherein the case is made of silicone polymer.

4. The wearable hand sanitizer dispenser of claim 1, wherein the pocket is removably attached to the body by fastening means.

5. The wearable hand sanitizer dispenser of claim 1, wherein the pocket is an integral part of the body.

6. The wearable hand sanitizer dispenser of claim 1, wherein the openings on the pocket are self-sealing slits.

7. The wearable hand sanitizer dispenser of claim 1, wherein the openings on the case are self-sealing slits.

8. The wearable hand sanitizer dispenser of claim 1 further comprises means to control releasing of sanitizer and to prevent sanitizer from leakage including but not limited to diaphragm valves and check valves.

9. The wearable hand sanitizer dispenser of claim 1, wherein the refill apertures on the case may further have caps.

10. The wearable hand sanitizer dispenser of claim 1, wherein the pocket may be a pouch like receptacle with an opening to insert and take out the silicon encased sponge.

11. A wearable hand sanitizer dispenser comprising:
a. a body member with holes;
b. a pocket located on the body member, the pocket either removably attached to the body by fastening means or being an integral part of the body, the pocket having a plurality of self-sealing slits for controlling release of sanitizer;
c. a silicone case located within the pocket, the silicone case having a plurality of self-sealing slits for controlling release of sanitizer and a plurality of refill apertures for refilling of sanitizer,
d. a sponge encased in the silicone case for absorbing sanitizer; and
e. an interjecting means for interjecting sanitizer into the silicone case.

12. The wearable hand sanitizer dispenser of claim 11, wherein the refill apertures on the silicone case may further have caps.

13. The wearable hand sanitizer dispenser of claim 11 further comprises means to control release of sanitizer and to prevent sanitizer from leakage including but not limited to diaphragm valves and check valves.

14. The wearable hand sanitizer dispenser of claim 11, wherein the pocket may be a pouch like receptacle with an opening to insert and take out the silicon encased sponge.
